(19) United States
(12) Patent Application Publication
(10) Pub. No.: US 2003/0211454 A1
(43) Pub. Date: Nov. 13, 2003

(54) Detection reagent

(76) Inventors: Nicholas Thomas, Cardiff (GB); Michael F. Cooper, Cardiff (GB); Elaine Adie, Cardiff (GB)

Correspondence Address: AMERSHAM BIOSCIENCES, PATENT DEPARTMENT, 800 CENTENNIAL AVENUE, PISCATAWAY, NJ, 08855, US

(21) Appl. No.: 10/182,994

(22) PCT Filed: Feb. 1, 2001
(86) PCT No.: PCT/GB01/00402

(30) Foreign Application Priority Data
Feb. 2, 2000 (GB) 0002261.6
Dec. 21, 2000 (GB) 0031168.8

(51) Int. Cl. C12Q 1/00; C 07D 4 3/02

(52) U.S. Cl. 435/4; 546/176; 548/454

(57) ABSTRACT

Disclosed is an environmentally sensitive ratiometric reporter molecule. The molecule is a compound of Formula (I) wherein D₁ and D₂ are detectable molecules and D₁ is a reference molecule; D₂ is an environmentally sensitive molecule; and L is a linker group.

The present invention relates to environmentally sensitive reagents. In particular, the present invention relates to environmentally sensitive ratiometric probes.

Many detectable molecules are generally known to be used for labelling and detection of various biological and non-biological materials in the study of biological processes. A number of such molecules are sensitive to their environment and may, therefore, be used as indicators to measure environmental conditions such as intracellular or extracellular changes.

In particular, fluorescent dye molecules are used in techniques such as fluorescence microscopy, flow cytometry and fluorescence spectroscopy and a number of such dyes are sensitive to their environment giving different fluorescent signals depending on the presence or absence of environmental signals.

For example, a number of fluorescent probes are available which have different fluorescence properties depending on the pH of their immediate environment. Intracellular pH is generally between approximately 6.8 and 7.4 in the cytosol and approximately 4.5 and 6.5 in the cell's acidic organelles. The pH inside a cell varies by only fractions of a pH unit and such small changes can be quite slow. pH changes have been implicated to be involved in a diverse range of physiological and pathological processes. For example, a cytosolic pH change of pH 7 to pH 6.5 and a mitochondrial change of pH 7.2 to 8.0 have been measured in apoptosis. pH changes have also been measured in cell proliferation, muscle contraction, endocytosis, malignancy and chemotaxis disease (see, for example, Martinez-Zaguilan R, Gillies R J (1996) Cell Physiol Biochem 6:169-184; Okamoto C T (1998) Adv Drug Deliv Rev 29:215-228; Falke J J, Bass R B, Butler S L, Chervitz S A, Danielson M A (1997) Ann Rev Cell Dev Biol 13:457-512; Shimizu Y, Hunt III S W (1 996) Immunol Today 17:565-573).

External pH changes can also give an indication of cellular changes For example apoptosis of cells in a sample can be detected by an increase in extracellular pH. Similarly, lysosomal secretion can be detected by extracellular pH changes.

There are also a number of fluorescent dyes commercially available which will measure calcium levels using a number of excitation and emission wavelengths such as Fura 2, Fluo-3, Fluo-4 and Quin2. These can be used to measure calcium ion flux which may be stimulated in a variety of ways within a cell. During such a process intracellular free Ca²⁺ concentrations can change rapidly by as much as 100 fold (Nuccitelli R (1994) A Practical Guide to the Study of Calcium in Living Cells Vol 40 Academic press San Diego USA). The known probes generally have altered fluorescence properties according to whether they are in a Ca²⁺-bound or unbound state.

Other specific ion sensors can be used to detect extracellular or intracellular ion concentrations. For example, a general charge sensing probe like DiBAC₄ (see, for example, Rink T J, Tsien R Y, Pozzan T (1982) J Cell Biol 95:189-196) can be used to measure ionic gradients across membranes. Increases and decreases in membrane potential—referred to as membrane hyperpolarisation and depolarisation, respectively—play a central role in many physiological processes, including nerve-impulse propagation, muscle contraction, cell signalling and ion-channel gating.

Measurement of other ions of interest including K⁺, Na⁺, Cl⁻, Mg²⁺, Zn²⁺ and other heavy metal ions is also desirable. There are a variety of probes available which will detect such ions e.g. Sodium Green, Magnesium Green, Calcium Crimson, Mag-Fluo-4, Newport Green (K⁺), N-(6-methoxy-8-quinoyl)-p toluenesulfonamide (TSQ for Zn²⁺), PhenGreen PL (Cu²⁺), SPQ(6-methoxy-N-(3-sulfopropyl)quinolinium inner salt for Cl⁻ detection). 1,2-diaminoanthraquinone is used for the quantification of NO and NO₂⁻.

Fluorescent probes can also be used in enzyme-substrate assays such as assays for proteases, kinases, transferases, or to detect protein-protein interactions. In such assays, the fluorescent probes themselves may be modified by enzyme activity leading to a change in fluorescent properties of the probe. For example there are phosphate probes which can detect the activity of kinases and phosphatases e.g. 7-hydroxy-9H-(1,3-dichloro-9,9-dimethylacridone-2-one) (DDAO), resorufin (available from Molecular Probes Inc.).

However, the use of detectable molecules such as these dyes in biological systems is subject to a number of problems which may make the results obtained difficult to interpret. For example, where a dye is transported into a cell to measure an intracellular concentration of ions, there may well be variable uptake of the dye itself or variation in the size of the cell (such that a larger cell may have a higher concentration of probe). Thus, a high fluorescence in one particular cell when compared to another may not be through an increased ion concentration or other environmental signal alone, but may be a result of cell size, permeability or stage of the cell cycle, for example. In addition, fluor quenching may occur when probes are in close proximity (this is particularly important when, for example, a pH sensitive dye is internalised into acidic vesicles where the dye is perhaps more concentrated than when it is on the cell surface).

It is therefore important, when looking at the translocation of a detectable molecule such as a fluorescently labelled compound within the cell, that there is a constant marker which will act as an internal standard compensating for concentration dependent effects in fluorescence. Accordingly, ratiometric probes have been developed which allow a constant and a variable signal to be detected, the variable signal changing according to the environmental conditions. By measuring changes in the ratio of the two signals, the measurement of signal from the environmentally sensitive moiety can be made independent of the amount of uptake of the probe or the size of the cell. That is, ratiometric probes allow concentration independent measurements to be made. This allows more precise measurements and, with some probes, quantitative detection is possible.

Current ratiometric probes include SNARF®, SNAFL®, LysoSensor™ and LysoTracker™ Yellow/Blue/Red (Molecular Probes, Inc.), all of which are used for making pH measurements. However, these probes generally comprise a single fluorescent entity and interpreting their fluorescence signals in different environmental conditions requires the resolution of complex spectra from that single entity. The change in emission in these probes at different pH is detected over a relatively small range of wavelengths. Moreover, SNARF® and SNAFL® have decreased fluorescence in acidic conditions and increase their fluorescence at neutral pH. Because of this, these probes are not useful for measuring membrane internalisation (mediated by a cell surface receptor or other means) as both produce a signal decrease on internalisation. Other probes such as LysoSensor™ and LysoTracker™ lack functionalisation so cannot be conjugated to particular biological molecules of interest.

Accordingly there is a need for improved ratiometric probes to be developed.

One object of the present invention is to provide a ratiometric reporter molecule by linking two moieties, one of which is a reference molecule providing an approximately constant read-out, the other is an environmentally sensitive molecule which provides a variable reporting signal. The variable molecule may be a fluorescent probe which is sensitive to the local environment, i.e. its emission spectra may be effected by pH, ion concentration or some other measurable change. By relating the output of both probes a ratiometric read-out is produced. This approach can, therefore, eliminate diffusion and concentration factors when monitoring the local environment around the probe for changes whilst the use of two linked moieties with spatially separated spectra reduces the complex resolution of different spectra required when using the current ratiometric probes.

Accordingly, in a first aspect of the invention, there is provided a compound of Formula I:

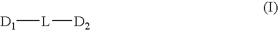

wherein D₁ and D₂ are detectable molecules and:

D₁ is a reference molecule;

D₂ is an environmentally sensitive molecule; and

L is a linker group.

Suitably, the reference molecule or the environmentally sensitive molecule may be detectable by any suitable detection method such as calorimetric, fluorescence, phosphoresence, luminescence, IR, Raman, NMR or spin label detection. In another embodiment, the appropriate detection method for D₁ and D₂ need not be the same.

In a particularly preferred embodiment of the first aspect of the invention, there is provided a compound of Formula I:

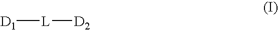

wherein D₁ and D₂ are detectable fluorophores and:

D₁ is a reference molecule;

D₂ is an environmentally sensitive molecule; and

L is a linker group;

characterised in that there is essentially no energy transfer between D₁ and D₂.

Suitably, detectable molecules D₁ and D₂ are fluorophores selected such that their emission spectra are spatially separated. D₁ and/or D₂ may be selected from fluoresceins, rhodamines, coumarins, BODIPY™ dyes and cyanine dyes. In a particularly preferred embodiment, D₁ and/or D₂ may be a cyanine dye. The Cyanine dyes (sometimes referred to as “Cy dyes™”), described, for example, in U.S. Pat. No. 5,268,486, is a series of biologically compatible fluorophores which are characterised by high fluorescence emission, environmental stability and a range of emission wavelengths extending into the near infra-red which can be selected by varying the internal molecular skeleton of the fluorophore. Preferred fluorophores D₁ and/or D₂ are the cyanine dyes such as any of Cy2 to Cy7 or their derivatives. The excitation (Abs) and emission (Em) characteristics of the unmodified dye molecules are shown:

| | Flourescence | | |
|---|---|---|---|
| Dye | Colour | Abs (nm) | Em (nm) |
| Cy2 | Green | 489 | 506 |
| Cy3 | Orange | 550 | 570 |
| Cy3.5 | Scarlet | 581 | 596 |
| Cy5 | Far red | 649 | 670 |
| Cy5.5 | Near-IR | 675 | 694 |
| Cy7 | Near-IR | 743 | 767 |

Alternatively, D₁ and/or D₂ may be a luminescent molecule such as a fluorescent or a bioluminescent protein, such as Green fluorescent protein (GFP) and analogues thereof.

Suitably, a “reference” molecule, D₁, is one which does not change its fluorescence properties in the presence of the environmental conditions to be detected by the reporter molecule of Formula I, while an “environmentally sensitive” molecule, D₂ is one which changes its fluorescence properties in the presence of the environmental conditions to be detected. Accordingly, introduction of the compound of Formula I into the appropriate environmental conditions will lead to a change in the emission spectra of the environmentally sensitive molecule while the reference molecule provides a constant readout. Thus the ratio of fluorescence emission from D₁ and D₂ when the molecule of Formula I is excited and monitored at two different wavelengths will change according to the environmental conditions. It is particularly preferred that D₁ and D₂ are chosen such that the use of dual excitation wavelengths and dual emission wavelengths allows the fluorescence from the two linked probes to be observed at spatially separated wavelengths and, thus, allowing ratiometric measurements to be made synchronously. In a particularly preferred embodiment, therefore, the excitation wavelength of D₁ is different to the excitation wavelength of D₂ such that, one of D₁ or D₂ has a higher excitation wavelength than the other.

Detectable environmental conditions include changes in pH, changes in ion concentrations and presence of enzyme.

Suitably, the environmentally sensitive molecule, D₂, is selected from dyes that change fluorescence due to pH changes such as pH sensitive Cy dyes (Cooper et al. J. Chem. Soc. Chem. Comm. 2000, 2323-2324), dyes that change fluorescence due to enzyme activity, dyes that change fluorescence due to ion concentrations (such as chelating dyes, Fura 2, Fluo-3, Fluo-4, Quin2, Sodium Green, Magnesium Green, Calcium Crimson, Mag-Fluo-4, Newport Green (K⁺), N-(6-methoxy-8-quinoyl)-p toluenesulfonamide (TSQ for Zn²⁺), PhenGreen PL (Cu²⁺), SPQ(6-methoxy-N-(3-sulfopropyl)quinolinium for Cl⁻ detection), 1,2 diaminoanthraquinone and DiBAC₄ and dyes that change fluorescence due to covalent modifications e.g. phosphorylation, lipid modifications and so forth.

D₂ may also be a known fluorescent dye that has been modified to change its properties according to specific environmental conditions. Suitably, D₂ can be modified by inclusion of a group that acts as an enzyme substrate such that the fluorescence properties of D₂ are affected by the presence of the enzyme.

The linker group, L, may be characterised as a chemical adduct that covalently links both D₁ and D₂.

Preferably, this may act as a group that maintains the two dyes within a finite distance whilst having no effect on the spectroscopic properties of the dyes. Keeping the probes within a finite distance allows spectral comparisons between the probes to be made as a function of concentration and thus allows ratiometric measurement.

The linker group may act to hold two distinct dyes capable of energy transfer in a particular orientation so that the dipole-dipole interactions of the two dyes, and thus energy transfer, are minimised, and the dyes act independently of each other.

Suitable linking groups, L, include amino acids, such as lysine or ornithine, which contain several labelling sites that can be masked using protecting group chemistry thus allowing site specific labelling of the amino acid and the build up of a tandem cassette in a step-wise fashion. Suitable labelling sites include amines. In one embodiment, linking groups are poly-amino acids such as polyproline which may, preferably, comprise from 6 to 12 proline units.

Alternatively, the linker group may act to maintain two dyes that are capable of energy transfer at a finite distance that is very much greater than Rₒ. where Rₒ is the Förster radius i.e. the distance between two fluors where the efficiency of energy transfer is equal to 50%, and therefore energy transfer does not occur. Rₒ values are typically within a range of 30-60 Angstroms.

Linkers may also be rigid thus holding the probes in an orientation that restricts collisional quenching. This may include linkers such as polyproline residues or steroidal linkers.

The linker group for reporter compound of Formula I may also act to hold two probes within a finite distance but energy transfer from one dye to another is restricted, due to the emissive excited states being of different spin parity. For example the pairing of an excited singlet state dye with an excited triplet state dye results in that the two probes are incompatible for Förster energy transfer i.e. are parity forbidden and therefore not able to transfer or accept excited state energy.

In a particularly preferred embodiment, the linker, L, may also include a reactive group that can be conjugated to a biomolecule such as an antibody, protein, peptide or oligonucleotide. Suitable groups include N-hydroxy succinimides, isothiocyanates, maleimides, iodoacetamides and hydrazides.

Suitably, linker group L may be from 2-30 bond lengths. For example, if the linker group contains an alkyl chain, —(CH₂)ₙ—, the carbon number “n” may be from 1 to about 15. The linker group may include part of the constituents extending from the fluorochrome. In other words, the linker group is attached to the dye chromophore but is not a part of it.

Suitable linking groups are non-conjugated groups which may be selected from the group consisting of alkyl chains containing from 1 to 20 carbon atoms, which may optionally include from 1 to 8 oxygen atoms as polyether linkages, or from 1 to 8 nitrogen atoms as polyamine linkages, or from 1 to 4 CO—NH groups as polyamide linkages.

Methods for covalently linking fluorochromes through a linker group are well known to those skilled in the art.

For example, where the linker group contains an amide or an ester, a ratiometric reporter molecule may be prepared by the reaction of a compound of formula (V) with a compound of formula (VI);

| | |
|---|---|
| R-(M)-COA | B-(N)-R |
| (V) | (VI) |

wherein R and R′ are different fluorochromes; COA is an activated or activatable carboxyl group; B is NH₂ or OH; and M and N are independently aliphatic moieties containing C₁₋₁₂ alkyl and optionally including one or more linking phenyl, napthyl, amide, ester, or ether functionalities. See for example, Mujumdar, R. B. et al. Bioconjugate Chemistry, Vol. 4, pp 105-111, (1993); and U.S. Pat. No. 5,268,486. Suitable groups A include halo, for example chloro or bromo, para-nitrophenoxyl, N-hydroxysuccinimido, or OCOR″ wherein R″ is C₁₋₆ alkyl.

Complexes of the present invention wherein the linker group contains an amino, ether or thioether group, may be prepared by the reaction of a compound of formula (VII) with a compound of formula (VIII);

| | |
|---|---|
| R-(M)-B′ | C-(N)-R′ |
| (VII) | (VIII) |

wherein R, R′, M and N are as defined above; B′ is OH, NH₂, or SH; and C is a displaceable group for example iodo, or para-toluenesulphonate. The reaction is suitably carried out in the presence of a base.

In another embodiment, the linker may be cleavable, for example. chemically cleavable, photocleavable (e.g. nitrobenzylalcohol) or enzymatically cleavable (e.g. ester, amide, phosphodiester, azo) by enzymes such as proteases. Suitable methods for cleaving such a linker are well known and described, for example, in Gerard Marriott et al., Preparation and photoactivation of caged fluorophores and caged proteins using a new cross-linking reagent, Bioconjugate Chemistry; (1998); 9(2); 143-151.

Energy transfer is the transfer of excited state energy between two probes that are within a short distance of each other. This may occur by Förster energy transfer, by collisional transfer, where energy transfer occurs from an electronically excited molecule to a ground state molecule, or where a photon is emitted and reabsorbed between two molecules in short range e.g. two contiguous dyes.

By “essentially no energy transfer” it is meant that D₁ and D₂ are chosen and linked such that the amount of energy transfer between the two is minimal. Preferably, D₁ and D₂ have spectroscopic characteristics i.e. excitation and emission spectra such that there is essentially no overlap between the emission spectrum of one and the absorption spectrum of the other. Thus, the amount of transfer between the two components is minimal. In one embodiment, the amount of energy transfer between the two components is approximately 25% or below. In a preferred embodiment, the amount of transfer between the components is approximately below 10%.

In a preferred embodiment, the compound of Formula I may be pH sensitive and, therefore, suitable for the measurement of agonist-induced internalisation of cell surface receptors which is facilitated via an acid vesicle. This can be performed in several ways. One of these ways is by labelling the cell surface (of a cell expressing a particular receptor) with the compound of Formula I, via a reactive ester, such as NHS for example, or by other means, and then treating the cell with an agonist or other ligand which will induce internalisation of the receptor. The compound of Formula I will thus be internalised on agonist treatment and the internalisation assessed through changes in the pH leading to modifications to the fluorescent properties of component D₂. Fluorescence measurements of D₁ will monitor any concentration (or other) dependent changes in fluorescence and allow a ratiometric measurement to be collected. Another way of measuring agonist-mediated dye internalisation is in a receptor-specific manner. The cell surface receptor in question can be analysed by labelling it directly with a compound of Formula I which is, preferably, pH sensitive. Labelling can be achieved. for example, by using a labelled antibody directed towards a receptor specific epitope and then treating the cell with agonist or ligand to induce internalisation. Antagonist effects can be measured by direct competition experiments. In another embodiment the ligand acting on the receptor can be labelled with the dye, and internalisation monitored by the change in pH as the ligand is internalised alongside the receptor.

Accordingly, in a particularly preferred embodiment, the compound according to the first aspect of the invention is a compound of Formula II

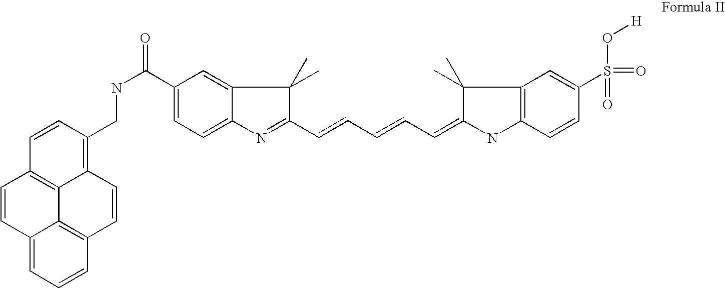

In this embodiment, the reference molecule D₁ is pyrene while the environmentally sensitive molecule D₂ is a pH sensitive Cy5 dye (pKa=6.1 in water) which is sensitive to changes in pH. The linker group L is a methyl-amide link, CH₂—NH—CO.

In another embodiment of the first aspect, the compound of Formula I will be suitable for making measurements of enzyme activity, suitably nitroreductase enzyme activity.

The bacterial enzymes termed nitroreductases have been shown to catalyse the general reaction set out below in Reaction Scheme 2:

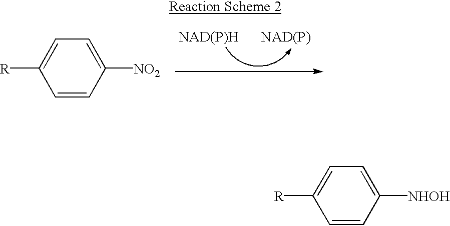

where, in the presence of NADH or NADPH, one or more —NO₂ groups on an organic molecule are reduced to a hydroxylamine group which may subsequently be converted to an amine group.

Cy-Q or “dark dyes” are described in WO 99/64519. The change in fluorescence which arises from nitroreductase action on Cy-Q dyes can be exploited in the construction of ratiometric fluorescence reporters of Formula I wherein D₂ is a Cy-Q dye.

The structure-defined emission characteristics of the Cy-Q make it suitable for inclusion in a paired fluorophore ratiometric reporter compound of Formula I, where nitroreductase action on the Cy-Q leads to a change in the ratio of fluorescence emission from the paired fluors when excited and monitored at two different wavelengths. Such a ratiometric reporter molecule allows measurement of enzyme activity to be made independent of the concentration of the reporter molecule.

Accordingly, in one embodiment of the invention nitroreductase enzyme activity on D₂ leads to a change in the ratio of fluorescence emission from the compound of Formula I when excited and monitored at two different wavelengths.

In a particularly preferred embodiment, D₁ is a Cy dye molecule and D₂ is a Cy-Q molecule. Preferably, D₁ is Cy2 and D₂ is Cy5-Q such that the paired fluorophore comprises Cy2/Cy5-Q (Cy2 Abs 489/Em 506; Cy5-Q Abs 649/Em-; Cy5 Abs 649/Em 670).

In another preferred embodiment, a compound of Formula I or Formula II is permeable to cells. Preferably, the compound of Formula I or Formula II further comprises a cell membrane permeabilising group. Membrane permeant compounds can be generated by masking hydrophilic groups to provide more hydrophobic compounds. The masking groups can be designed to be cleaved from the fluorogenic substrate within the cell to generate the derived substrate intracellularly. Because the substrate is more hydrophilic than the membrane permeant derivative it is then trapped in the cell. Suitable cell membrane permeabilising groups may be selected from acetoxymethyl ester which is readily cleaved by endogenous mammalian intracellular esterases (Jansen, A. B. A. and Russell, T. J., J.Chem Soc. 2127-2132 (1965) and Daehne W. et al. J.Med-.Chem. 13, 697-612 (1970)) and pivaloyl ester (Madhu et al., J. Ocul. Pharmacol. Ther. 1998, 14, 5, pp 389-399) although other suitable groups will be recognised by those skilled in the art.

In a second aspect of the invention there is provided a method for detecting a change in environmental conditions.

Suitably said method comprises the steps of:

a) measuring the fluorescence emission of a compound of Formula I in the presence or suspected presence of the environmental signal to be detected; and

b) comparing with the fluorescence emission of the compound of Formula I in the absence of said environmental signal.

In a preferred embodiment, excitation of a ratiometric reporter compound of Formula I is with light of two different wavelengths, λ1 and λ2, where the wavelengths are chosen to be suitable to elicit fluorescence emission from the fluorophore D₁ and the fluorophore corresponding to D₂. This excitation yields fluorescence emission from D₁ at wavelength λ3 but yields only low or zero emission from D₂ at wavelength λ4. Subsequent reaction of the ratiometric reporter in the presence of the appropriate environmental signal, e.g. pH, ion concentration, enzyme activity etc., on D₂ yields an altered (either increased or decreased) fluorescence emission at λ4. Under these conditions, determination of the ratio of intensity of λ3:λ4 and comparison with the λ3:λ4 ratio of the unreacted reporter gives a measure of the degree of conversion of the ratiometric reporter into a molecule comprising the reduced form of D₂, and hence gives a measure of the presence of the relevant environmental signal.

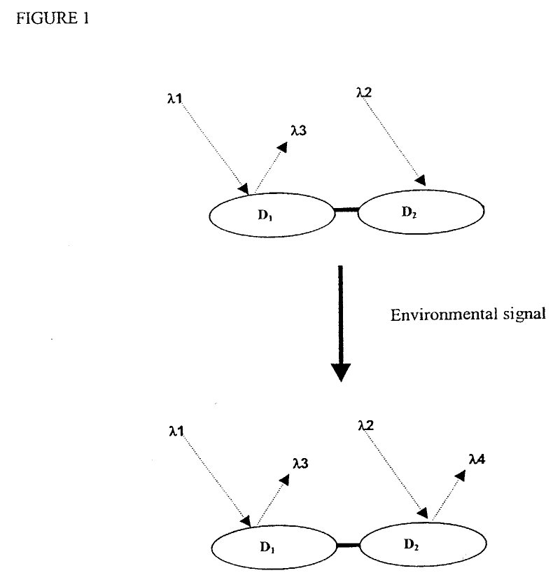

This is summarised in Reaction Scheme 1 (FIG. 1).

Accordingly, in a preferred embodiment of the second aspect there is provided a method comprising the steps of:

a) exciting a compound of Formula I with light of two different wavelengths, λ1 and λ2, where the wavelengths are chosen to be suitable to elicit fluorescence emission from the fluorophore D₁ and the fluorophore corresponding to D₂;

b) measuring fluorescence emission from D₁ at wavelength λ3 and fluorescence emission from D₂ at wavelength λ4

c) introducing the compound of Formula I to the appropriate environmental signal;

d) repeating excitation step a) and measurement step b);

e) determining the ratio of intensity of λ3:λ4 and comparing it with the λ3:λ4 ratio of the compound of Formula I in the absence of the environmental signal.

Measurement of fluorescence may be readily achieved by use of a range of detection instruments including fluorescence microscopes (e.g. LSM 410, Zeiss), microplate readers (e.g. CytoFluor 4000, Perkin Elmer), confocal microscopes, CCD imaging systems (e.g. LEADseeker™, Amersham Pharmacia Biotech) and Flow Cytometers (e.g. FACScalibur. Becton Dickinson). Recent developments in detection technologies allow rapid simultaneous emission and excitation measurements (see, for example, WO 99/47963). One example is the LEADseeker™ Cell Analysis System (Amersham Pharmacia Biotech) which allows the simultaneous excitation of multiple dyes, at distinguishable wavelengths, which are associated with cells or beads. The presence of multiple CCD cameras allows the detection of multiple emission wavelengths from these same dyes. Accordingly, in a particularly preferred embodiment of the second aspect, simultaneous dual excitation will be used. Suitable systems for simultaneous dual excitation include the LEADseeker™ Cell Analysis System.

In a preferred embodiment the fluorescence emission may be monitored continually over time in order to follow changes in environmental conditions over time.

In one embodiment of any of the previous aspects of the invention, increased fluorescence of the cyanine dye molecule is identified by analysis of fluorescence emission in the range 500 to 900 nm and, more preferably, 665-725 nm.

In one embodiment, the composition in which the environment is to be tested comprises a cell or cell extract. In principle, any type of cell can be used i.e. prokaryotic or eukaryotic (including bacterial, mammalian and plant cells). Where appropriate, a cell extract can be prepared from a cell, using standard methods known to those skilled in the art (Molecular Cloning, A Laboratory Manual 2^{nd} Edition, Cold Spring Harbour Laboratory Press 1989), prior to measuring fluorescence.

Cell based assays are increasingly attractive over in vitro biochemical assays for use in high throughput screening (HTS). This is because cell based assays require minimal manipulation and the readouts can be examined in a biological context that more faithfully mimics the normal physiological situation. Cell-based assays used in a primary screen provide reliable toxicological data whereby an antagonist can be distinguished from compounds that are merely just toxic to the cell. Such in vivo assays require an ability to measure a cellular process and a means to measure its output. For example, a change in the pattern of transcription of a number of genes can be induced by cellular signals triggered, for example, by the interaction of an agonist with its cell surface receptor or by internal cellular events such as DNA damage. The induced changes in transcription can be identified by fusing a reporter gene to a promoter region which is known to be responsive to the specific activation signal.

In fluorescence-based enzyme-substrate systems, an increase in fluorescence gives a measure of the activation of the expression of the reporter gene.

Typically, to assay for the presence of certain environmental conditions and, therefore, the activity of an agent to activate cellular responses via the regulatory sequence under study, cells may be incubated with the test agent, followed by addition of a cell-permeant ratiometric reporter molecule of Formula I. After an appropriate period required for conversion of the reporter molecule to a form showing different fluorescence properties, the fluorescence emission from the cells is measured at a wavelength appropriate for the chosen reporter.

The measured fluorescence is compared with fluorescence from control cells not exposed to the test agent and the effects, if any, of the test agent on gene expression modulated through the regulatory sequence is determined from the ratio of fluorescence in the test cells to the fluorescence in the control cells.

Where appropriate, a cell extract can be prepared using conventional methods.

For the purposes of clarity, certain embodiments of the present invention will now be described by way of example with reference to the following figures:

FIG. 1 shows Reaction Scheme 1, a schematic diagram of a ratiometric reporter molecule.

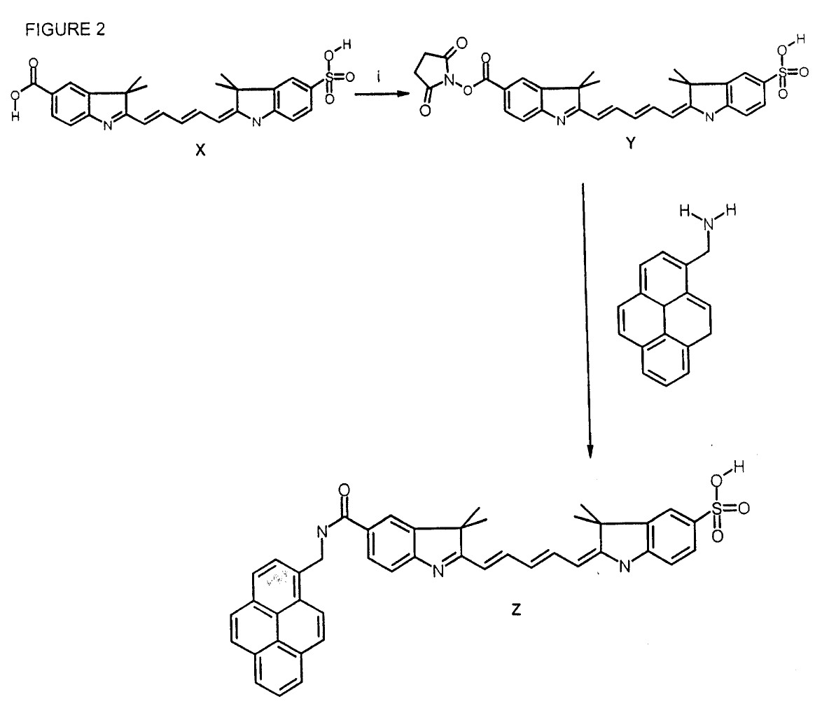

FIG. 2 shows Reaction Scheme 2, a reaction scheme for the synthesis of a non-energy transfer tandem dye cassette.

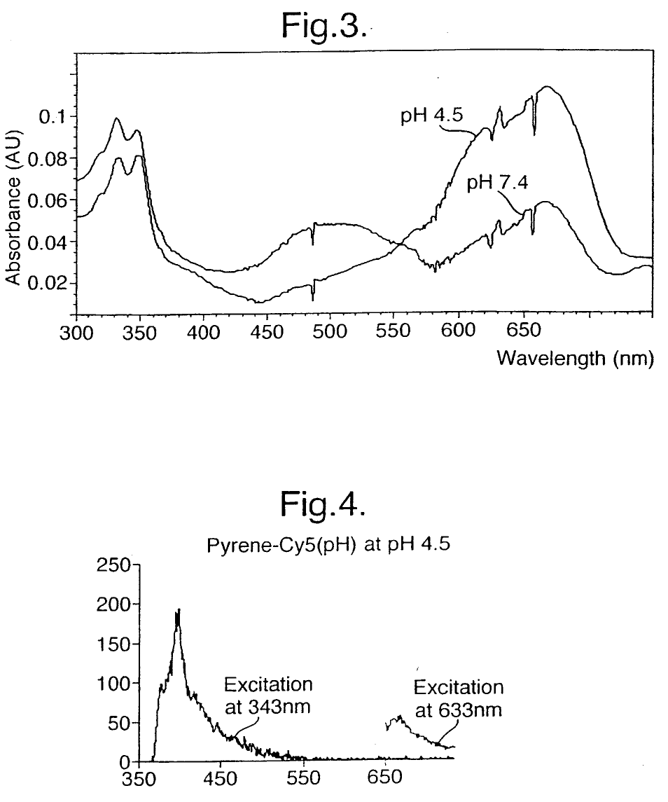

FIG. 3 shows UV/Visible absorption spectra of compound Z at pH 4.5 and pH 7.4.

FIG. 4 shows the emission spectra of compound Z at pH 4.5.

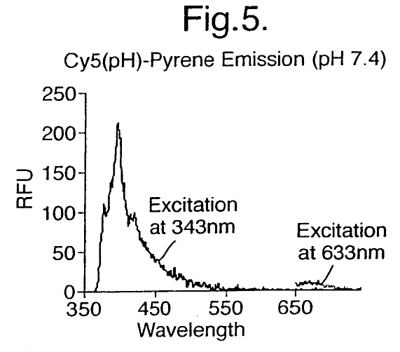

FIG. 5 shows emission spectra of compound Z at pH 7.4

### EXAMPLE 1

### Synthesis of a pH Sensitive Ratiometric Reporter Molecule

FIG. 2 shows Reaction Scheme 2 which is a reaction scheme for the synthesis of a pH sensitive tandem dye cassette.

a) Synthesis of Compound X (2-[1E,3E)-5-(3,3-dimethyl-5-sulfo-1,3-dihydro-2H-indol-2-ylidene)-1,3-pentadienyl]-3,3-dimethyl-3H-indole-5-carboxylic Acid)

5-Sulfo-2,3,3-trimethylindolenine (69.3 mg, 0.27 mmol), malonaldehyde bis(phenylimine) monohydrochloride (70 mg, 0.27 mmol) benzoic acid (66 mg 0.54 mmol) and benzoic anhydride (122 mg, 0.54 mmol) were dissolved in DMF (2 ml) and the solution was stirred for 10 minutes at 60° C. A solution of 2,3,3,-trimethylindolenium-5-carboxylic acid (47.4 mg, 0.27 mmol) in DMF (0.5 ml) was added and the reaction mixture heated at 60° C. for a further four hours. The resulting blue solution was cooled and purified by reverse phase HPLC using a Rainin Dynamax 60 Å C18 column at 10 ml/min with a solvent gradient of 15% B for 5 minutes ramping from 15% to 50% B over 75minutes, where A=H₂O (0.1% acetic acid) and B=acetonitrile (0.1% acetic acid). The retention time of XII was 55.4 minutes (UV/Vis. detection at 650 nm). Yield 74 mg, 58%. ¹H-NMR, (d₆-DMSO), δ 8.67 (m, 1H, β-proton in bridge), δ 7.85 (m, 1H, β-proton in bridge) δ 7.79 (s, 1H, Ar—3H), δ 7.57 (d, 1H, Ar—5H), δ 7.47 (d, 1H, 5H—Ar,), δ 7.35 (s, 1H, 3H—A′), δ 7.31 (d, 1H, 6H—Ar,) δ 7.24 d, 6H—Ar) δ 6.99 (t, 1H, γ-proton in bridge), δ 6.32 (d, 1H, (α-proton in bridge δ 6.19 (d, 1H (α′-proton in bridge), (s, 12H, (—CH₃)₂). Accurate mass spectroscopy M-H⁻)=477.1456 for C₂₆H₂₅N₂O₅S

b) Synthesis of Compound Y; N-Hydroxy-succinimidyl Ester of Compound X

Compound X was dissolved in DMSO with Benzotriazole-1-yl-oxy-tris-pyrrolidino-phosphonium hexafluorophosphate (PyBOP) (1 eq), N-hydroxy-succinimide (1 eq) and diisopropylethylamine (1 eq) (step (i)). The solution was stirred for 1 hour to give quantitative conversion to the NHS ester by TLC analysis. The resulting blue solution was purified by reverse phase HPLC using a Rainin Dynamax 60 Å C18 column at 10 ml/min with a solvent gradient of 15% B for 5 minutes ramping from 15% to 20% B from 5 to 15 minutes, and 20% to 30% B from 15 to 25 minutes and 30% to 50% from 25 to 80 minutes, where A=H₂O (0.1% acetic acid) and B=acetonitrile (0.1% acetic acid). The retention time of the NHS ester was 45 minutes (UV/Vis. detection at 650 nm).Yield 100%. MALDI-TOF mass spectroscopy m/z=578 (100%) for C₃₀H₃₀N₃O₇S (M⁺+H).

c) Synthesis of Pyrene-1 Conjugate (Compound Z)

Compound Y was dissolved in DMSO with pyrene-methylamine (1 eq) and diisopropylethyamine (1 eq) (step (ii)) and the reaction stirred at room temperature for 3 hours. The solution was purified by reverse phase HPLC using the following conditions. The gradient was 15% B for 5 minutes, then 15% to 50% for 75 minutes, then 50% to 1005 b for 25 minutes, where A=H₂O (0.1% acetic acid) and B=acetonitrile (0.1% acetic acid). The unreacted Cy5 eluted at 45 minutes and Compound Z eluted at 88 minutes. TLC 20% methanol/dichloromethane observed 1 blue spot R_{f}=0.25. MALDI-TOF mass spectroscopy m/z=691 (100%) for C₄₃H₃₇N₃O₄. UV (H₂O/H⁺) λ_{abs}=330 nm, 343 nm, 650 nm. UV (H₂O/OH⁻) λ_{abs}=330 nm, 343 nm, 500 nm, 650 nm.

### EXAMPLE 2

### Spectroscopic Characteristics of Compound Z

The UV/Visible absorption profiles of Z were measured at two distinct pH. Two equimolar solutions of Z were made up (˜10⁻⁶M) in phosphate buffers of pH 4.5 and 7.4. These were allowed to equilibrate for 1 hour. The cuvettes were acid washed with 1M HCl, rinsed with distilled deionised water and dried between each measurement. UV and visible absorption measurements were performed upon a Hewlett Packard 8453 UV/vis spectrophotometer with a diode array detector. Data were collected using an HP Vectra XA PC and analysed using HP 845x UV/Vis software.

FIG. 3 shows the UV/Visible absorption spectra of Compound Z at pH 4.5 and 7.4.

### EXAMPLE 3

### Fluorescence Emission Spectra of Compound Z in Acid and Base

The fluorescence characteristics of Z were characterised using a Perkin-Elmer LS50B in fluorescence mode using 10 nm excitation and emission slit widths. All measurements were performed in a 2 ml quartz cuvette of 1 cm pathlength. The cuvettes were acid washed with 1M HCl, rinsed with distilled deionised water and dried between each measurement.

All spectra were collected using a Gateway 2000 PS-120 PC and analysed using Perkin-Elmer Winlab software. Two equimolar solutions of Z were made up (˜10⁻⁶M) in phosphate buffers of pH 4.5 and 7.4. These were allowed to equilibrate for 1 hour. The fluorescence emission spectra were measured using both an excitation wavelength of 343 nm (pyrene) and 633 nm (Cy5).

FIG. 4 shows the emission spectra of Compound Z at pH 4.5.

FIG. 5 shows the emission spectra of Compound Z at pH 7.4.

It can be seen from FIGS. 4 and 5 that upon excitation of Compound Z at 343 nm at pH 4.5 that there is no emission from the Cy5 at 650-700 nm. Therefore it is unlikely that energy transfer is occurring either by Förster mechanism or collisional ET. Furthermore when exciting probe Compound Z at 633 nm, emission occurs from the Cy5.

Upon exciting probe Compound Z at 343 nm at pH 7.4 the emission characteristics are unchanged and there is no energy transfer to Cy5 e.g. no signal at 650 nm and also the pyrene emission spectra is unchanged indicating that the pyrene emission is not quenched by the characteristic Cy5 absorption peak that has evolved at 500 nm at this pH. Furthermore, excitation of probe Compound Z at 633 nm in pH 7.4 buffer shows little emission from Cy5. This is expected as the fluorescent emission of the pH sensitive Cy5 probe at this pH is greatly reduced.

1. A compound of Formula I: 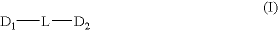
wherein D₁ and D₂ are detectable molecules and:
D₁ is a reference molecule;
D₂ is an environmentally sensitive molecule; and
L is a linker group.

2. A compound of Formula I: 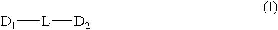
wherein D₁ and D₂ are detectable fluorophores and:
D₁ is a reference molecule;
D₂ is an environmentally sensitive molecule; and
L is a linker group;
characterised in that there is essentially no energy transfer between D₁ and D₂.

3. A compound as claimed in claim 1 or claim 2 wherein D₁ and D₂ have spectroscopic characteristics such that there is essentially no overlap between the emission spectrum D₁ and the absorption spectrum of D₂.

4. A compound as claimed in any of claims 1 to 3 wherein D₂ is selected from an environmentally sensitive Cy dye, Fura 2, Fluo-3, Fluo-4, Quin2, Sodium Green, Magnesium Green, Calcium Crimson, Mag-Fluo-4, Newport Green (K⁺), N-(6-methoxy-8-quinoyl)-p toluenesulfonamide (TSQ for Zn²⁺), PhenGreen PL (Cu²⁺), SPQ(6-methoxy-N-(3-sulfopropyl)quinolinium for Cl⁻ detection), 1,2 diaminoanthraquinone and DiBAC₄.

5. A compound as claimed in any of claims 1 to 4 wherein L is selected from amino acids which contain several amine labelling sites.

6. A compound as claimed in any of claims 1 to 5 wherein Rₒ is within a range of 30-60 Angstroms.

7. A compound as claimed in any of claims 1 to 6 wherein L further comprises a reactive group that can be conjugated to a biomolecule.

8. A compound as claimed in claim 7 wherein said reactive group is selected from N-hydroxy succinimides, isothiocyanates, maleimides, iodoacetamides and hydrazides.

9. A compound as claimed in any of claims 1 to 8 wherein L is a cleavable group.

10. A compound as claimed in claim 2 having Formula II:

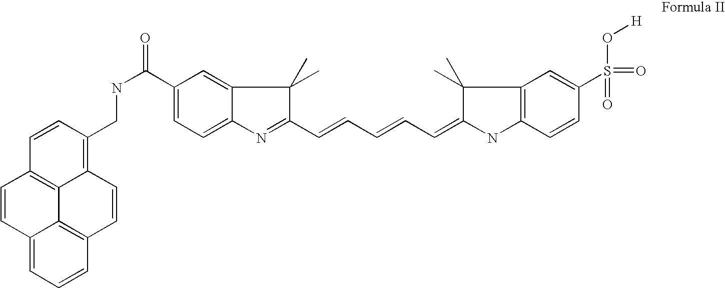

11. A compound as claimed in any of claims 1 to 10 wherein the compound is cell permeable.

12. A method for detecting a change in environmental conditions using a compound as claimed in any of claims 1 to 11.

13. A method as claimed in claim 12 comprising the steps of:
a) measuring the fluorescence emission of a compound of Formula I in the presence or suspected presence of the environmental signal to be detected; and
b) comparing with the fluorescence emission of the compound of Formula I in the absence of said environmental signal.

14. A method as claimed in claim 13 comprising the steps of:
a) exciting a compound of Formula I with light of two different wavelengths, λ1 and λ2, where the wavelengths are chosen to be suitable to elicit fluorescence emission from the fluorophore D₁ and the fluorophore corresponding to D₂;
b) measuring fluorescence emission from D₁ at wavelength λ3 and fluorescence emission from D₂ at wavelength λ4
c) introducing the compound of Formula I to the appropriate environmental signal;
d) repeating excitation step a) and measurement step b);
e) determining the ratio of intensity of λ3:λ4 and comparing it with the λ3:λ4 ratio of the compound of Formula I in the absence of the environmental signal.

15. A method as claimed in any of claims 12 to 14 wherein the measurement of fluorescence emission is by fluorescence microscopy, confocal microscopy, microplate reading, CCD imaging or flow cytometry

16. A method as claimed in claim 15 wherein excitation of D₁ and D₂ at distinguishable wavelengths is performed simultaneously.

17. A method as claimed in any of claims 12 to 16 wherein fluorescence emission is monitored continually over time to follow changes in environmental conditions.
